(19) United States
(12) Patent Application Publication
(10) Pub. No.: US 2005/0182324 A1
(43) Pub. Date: Aug. 18, 2005

(54) Methods of estimating ultrasound scatterer parameters in soft tissues

(76) Inventors: Bjorn A.J. Angelsen, Trondheim (NO); Tonni F. Johansen, Trondheim (NO)

Correspondence Address: Lance J. Lieberman, Esq.;Cohen, Pontani, Lieberman & Pavane, Suite 1210, 551 Fifth Avenue, New York, NY, 10176, US

(21) Appl. No.: 11/030,385

(22) Filed: Jan. 6, 2005

Related U.S. Application Data
(60) Provisional application No. 60/534,417, filed on Jan. 6, 2004.

(51) Int. Cl. A61B 8/00

(52) U.S. Cl. 600/437

(57) ABSTRACT

A method for characterizing ultrasound scatterers in soft tissue, wherein scattered signals are used to eliminate the absorption attenuation of an ultrasound wave from estimated parameters and to obtain directional scattering information. The scattered signals are obtained from either multiple regions where one region is used as a reference or multiple scattering directions, creating known reference signals from the same scattering region. The method provides new ultrasound parameters for the characterization and contrast enhancement of tissue structures in ultrasound imaging, such as tumor structures, ischemia of a myocardial wall and/or plaque compositions in vascular atheroma. The method of the invention can be used with various arrangements of ultrasound transducers, particularly switched linear arrays.

### RELATED APPLICATIONS

This application claims priority from U.S. Provisional Patent Application Ser. No. 60/534,417 which was filed on Jan. 6, 2004.

### BACKGROUND OF THE INVENTION

1. Field of the Invention

The present invention, relates to characterization of ultrasound scatterers in soft tissue. The method eliminates the effect of unknown ultrasound absorption between the transducer and the scatterers, and produces local scattering parameters of the tissue and their frequency dependence.

2. Description of the Related Art

Ultrasound back scatter imaging is a widely used imaging modality for diagnosis of many diseases of humans and animals. However, at present the method is used mainly to visualize anatomic structures, blood velocities, movements, and shear elasticity of tissue, and there is a further need for improved characterization of the scattering tissue structures with dimensions below the resolution in the anatomic image.

Transmitting on one transducer with the direction of the beam incident on the scatterers defined by the angle θₜ and receiving on the same or another transducer so that the angle between the transmit and the receive beams is θₜᵣ, one can write the frequency dependency of the received power of the scattered signal from the cross over region of the transmit and receive beams (105 of FIG. 1)
Sₜᵣ=K(Δβ−Δγ cos θₜᵣ)²VₛH(f,θₜᵣ;θₜ)f⁴e^{-2a(s)f} (1)
where K is an amplitude parameter, f is the ultrasound frequency, and e^{-2a(s)f} represents acoustic absorption where a(s) is the integral of the absorption coefficient for the whole propagation distance s of the signal along transmit and the receive beams. Δβ is the relative deviation in the bulk compressibility and □ □ is the relative deviation in the mass density between the scatterer and the surrounding medium, Vₛ is the scatterer volume, and H(f, θₜᵣ; θₜ) is a function that represents that scatterer size and shape and the dependency of the scattered intensity on the direction θₜ of the incident beam, and the angle θₜᵣ between the incident and receiving beams, for example θₜᵣ=θ₁₂ as illustrated in FIG. 1. For scatterers that are much smaller than the wavelength λ=c/f, where c˜1.54 mm/μsec is the ultrasound propagation velocity in the tissue, we have H(f, θₜᵣ; θₜ)=1. The scatterers are then referred to as point scatterers.

As the scatterer dimensions approaches or becomes larger than λ (for example as λ is reduced with higher frequencies), one gets interference from waves scattered at different regions of the scatterer. Depending on the angles θₜ and θₜᵣ, one can get destructive interference that reduces the scattered power in certain directions, or constructive interference that increases the scattered power in other directions. The invention provides methods on how one can use this angular variation to characterize the scatterers.

We hence notice that information about the acoustic parameters of the scatterers, Δβ and Δγ, and the scatter size and shape are found in the magnitude of the scattered intensity, i.e. (Δβ−Δγ cos θₜᵣ)²VₛH(f,θₜᵣ;θₜ), the frequency variation of the intensity, H(f,θₜᵣ;θₜ)f⁴e^{-2a(z)f}, and the variation of the scattered intensity with the directions of the incident and receive beams (Δβ−Δγ cos θₜᵣ)²H(f,θₜᵣ;θₜ).

With direct back-scattering, i.e. the transmit and receive transducers are the same so that θₜᵣ=0, we can often approximate the absorption integral as
a(s)=2az (2)
where z is the distance between the transducer and the scatterer so that s=2z, and a is an absorption parameter with typical values of 0.035-0.058 Neper/cmMHz. These values correspond to an absorption attenuation of 0.3-0.5 dB/cmMHz.

From Eq.(1) we see that absorption plays an important role in the frequency dependency of the scattered intensity, except when f is so low that the frequency variation of e^{-2a(s)f} is negligible compared to the other terms. To get some more insight into this, we do as an initial exercise assume that we have point scatterers (H(f,θₜᵣ;θₜ)=1), which gives the back scattered (i.e. ,θₜᵣ=0)
S_{back}=K(Δβ−Δγ)²Vₛf⁴e^{-2a(s)f} (3)

Differentiation with respect to f gives a maximum of this function, and also a maximum in the frequency variation of f⁴e^{-4azf}, for

$${f}_{0} = \frac{1}{az} \qquad ( 4 )$$

The absorption term will then have negligible effect on the frequency variation of the scattered intensity when f<<f₀, say f<0.2 f₀. From Eq.(4) we see that f₀ is approximately inversely proportional to the depth, where we shall analyze three examples.

For intravascular imaging (IVUS) of coronary plaque we have typically z˜0.2 cm, for noninvasive imaging of carotid plaque we have typically z˜2.5 cm, and for noninvasive imaging of liver disorders we have typically z˜7 cm. With the values of α given above, we get the following frequencies for peak of Eq.(3):
IVUS CoronaryPlaque: f₀=85-145 MHz
Noninvasive Carotid Plaque: f₀=7-12 MHz
Noninvasive Liver scatterers: f₀=2.5-4 MHz (5)

For the carotid and liver imaging, f₀=7-12 MHz covers the actual frequencies used for imaging, so that in these situations the absorption has a dominating effect on the frequency variation of the scattering. Hence, in these situations one should find methods for scatterer characterization, where the effect of frequency variation of absorption on the scattered intensity is reduced.

For IVUS imaging at 20-30 MHz, the influence of absorption on the frequency variation of the scattered intensity can be neglected when the scatterer dimension approaches or gets larger than the wavelength, which is 50-80 μm for these frequencies. Hence, the frequency variation of the scattered intensity can contain some information for scatterers with dimensions approaching ˜50 μm.

As the absorption is roughly proportional to the frequency, the image depth is inversely proportional to the frequency. For imaging of the carotid vessel and similar down to 40 mm depth, one generally uses 10 MHz ultrasound. Hence, IVUS imaging of coronary artery wall down to 4 mm is hence very attractive at ˜100 MHz. According to Eq.(5) the ultrasound absorption will then play an important role in the frequency dependency of the back scattered intensity. The wave length at 100 MHz is ˜15 μm, and by reducing the effect of absorption on the frequency variation of the scattered intensity, one is able to extract information on scatterers down to ˜2 μm dimension.

### SUMMARY OF THE INVENTION

The present invention provides methods for characterizing the scatterers in ultrasound imaging that strongly reduces the effect of absorption attenuation of the waves in the characterization, and makes it possible to eliminate the effect of frequency dependent absorption in the transmit path of the ultrasound pulse, and obtain frequency dependent scattering parameters from local regions in the tissue. It is furthermore possible to obtain information of scattering anisotropy in such regions, that can provide information about fiber direction in fibrous and muscular tissue, as well as degree of fibrosity. Moreover, by comparing ultrasound angular scattering with back scattering, one is able to derive anisotropic properties of the scattering cross section from a local region, that can give information of fibrous structures in the tissue.

The method provides new ultrasound parameters for characterization and contrast enhancement of tissue structures in ultrasound imaging, like tumor structures, ischemia of a myocardial wall, and plaque composition in vascular atheroma. It can be used with many arrangements of ultrasound transducers, particularly switched linear arrays.

The essence of the invention, is to use scattered signals either from multiple regions where one region is used as a reference, or multiple scattering directions, creating own reference signals from the same scattering region, so that the absorption attenuation of the ultrasound wave is eliminated from the estimated parameters, and directional scattering information can be achieved.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 shows an example embodiment according to the invention of two transducers arranged so that the scattering parameters of the tissue in the overlap region of the transducers beams can be investigated;

FIG. 2 shows example embodiments according to the invention where a switched linear array is used to realize two transducer apertures, which are arranged so that the scattering parameters' of the tissue in the overlap region of the apertures' beams can be investigated;

FIG. 3 illustrates how the scattering from tissue consisting of scatterers comparable or larger than the wavelength can be investigated using a linear array; and

FIG. 4 shows an application of the method described, imaging of anisotropic structures of a carotid plaque combined with two dimensional backscatter imaging.

### DETAILED DESCRIPTION OF THE PRESENTLY PREFERRED EMBODIMENTS

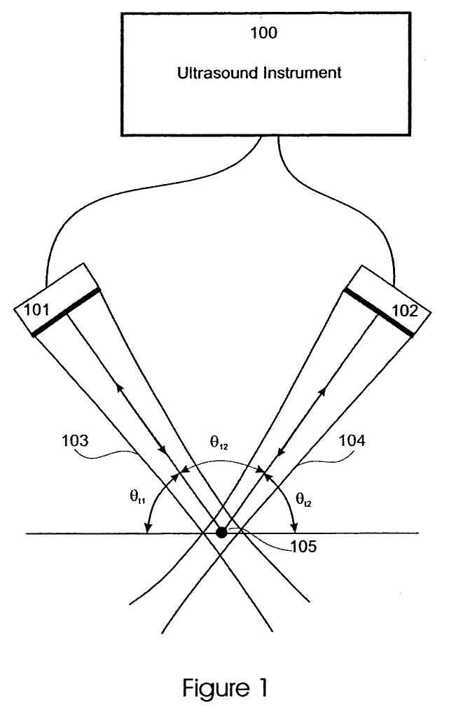

In the following we describe example embodiments of the invention with reference to the drawings. FIG. 1 shows two ultrasound transducers 101 and 102 that are connected to an ultrasound instrument 100 that allows selectable transmission of ultrasound pulses on each transducer, with selectable reception of the scattered signal on each transducer, independent of the transducer selected for transmission. The transducers can be directed at an angle towards each other, where θₜ represents the direction of the transmit beam and θₜᵣ is the angle between the transmit beam and the receive beam. In the Figure we have θₜ=θₜ₁ when transmitting on Transducer 1 (101) and θₜ=θₜ₂ when transmitting on Transducer 2 (102). We denote the beam 103 from Transducer 1 as Beam 1, and the beam 104 from Transducer 2 as Beam 2. The beams intercept in a region labeled 105 in FIG. 1. The angle between Beam 1 and Beam 2 is indicated as θ₁₂ in FIG. 1. When different beams are used to transmit and receive one gets θₜᵣ=θ₁₂, and when the same beam is used to transmit and receive one gets θₜᵣ=0.

A first method for reduction of the effect of frequency absorption according to the invention, is to use the scattered signal from reference scatterers with known frequency variation of the scattering cross section, and located in neighboring region with close to the same absorption as scatterers to be characterised. Such reference scatterers can be the erythrocytes in blood, where for characterization of arterial wall plaque one would use the signal scattered from the blood close to the plaque as reference. For other situations (like the liver) one would find the blood vessels close to the area of interest as reference, or use other reference scatterers in the neighborhood of the region of interest. In such situations one can do back scatter imaging and use the same beam directions for the transmit and receive beams. This will create a reference signal for back scatter imaging obtained from Eq.(3) as
S_{ref}˜σ_{ref}V_{ref}H_{ref}(f,0;θₜ)f⁴e^{-2a(2z)f} (6)
where σ_{ref}=(Δβ−Δγ)² for the parameters of the reference scatterers with back scattere imaging, and H_{ref} represents the frequency variation of the back scatterers for the reference scatterers. For point scatterers, like erythrocytes up to f˜10 MHz, H_{ref}=1.

The backscattered signal from blood is often masked in close to stationary reverberation noise. As the blood is moving, the scatterers also move and the backscattered signal from blood can be retrieved from the stationary noise by collecting back scattered signal from several pulses in substantially the same beam direction and perform high pass filtering of the signal along the pulse number coordinate. A small variation of the beam direction between the pulses can be accepted, as for example with continuous, mechanical scanning of the beam direction.

Ultrasound contrast agent micro bubbles can also conveniently be used as reference scatterers, both in visible blood vessels, and within the capillary vessels. The reference signal from the contrast agent bubbles can be separated from the signal from surrounding tissue by several known methods, such as harmonic imaging, pulse inversion imaging, and manipulation of the scattering properties by a low frequency pulse. Other reference scalterers can be identifiable cells, like normal liver cells, or fat cells in atheroma. The back scattered intensity from the reference scatterers can be approximated as
S_{scat}˜σ_{scat}V_{scat}H_{scat}(f,0;θₜ)f⁴e^{-2a(2z)f} (7)
σ_{scat}=(Δβ−Δγ)² for the scatterers in the actual region of the plaque, and V_{scat} is the volume of these scatterers. H(f,0;θₜ) then includes the frequency variation of the scattered signal due to the scatterer size and shape. The following ratio will then be independent of the absorption attenuation of the ultrasound wave along the beams

$$\frac{{S}_{scat}}{{S}_{ref}} \sim \frac{{σ}_{scat} {V}_{scat}}{{σ}_{ref} {V}_{ref}} \frac{{H}_{scat} ( f , 0 ; {θ}_{t} )}{{H}_{ref} ( f , 0 ; {θ}_{t} )} \qquad ( 8 )$$

A second method according to the invention, is to use angular scattering with two beams as illustrated in FIG. 1-4. First transmitting on Transducer 1 (101) and receiving the back scattered signal at a depth range along the beam corresponding to the cross over region 105 between the two beams, one receives a back scattered signal power on Transducer 1 and Transducer 2 (102) as
S₁₁=A₁²σ₁₁H(f,0;θₜ₁) Transmit Transd 1 and Receive Transd 1
S₁₂=A₁A₂σ₁₂H(f,θ₁₂;θₜ₁) Transmit Transd 1 and Receive Transd 2 (9)
σ₁₁=(Δβ−Δγ)² σ₁₂=(Δβ−Δγ cos θ₁₂)²
where A₁ contains the one-way power attenuation along Beam 1 (103) from Transducer 1 (101) to the scattering region 105, A₂ contains the one-way power attenuation along the Beam 2 (104) from Transducer 2 (102) to the scattering region 105. Transmitting at Transducer 2 (102) one gets scattered signal power from the overlap region (105) as
S₂₂=A₂²σ₂₂H(f,0;θₜ₂) Transmit Transd 2 and Receive Transd 2
S₂₁=A₂A₁σ₂₁H(f,θ₁₂;θₜ₂) Transmit Transd 2 and Receive Transd 1 (10)
σ₂₂=σ₁₁=(Δβ−Δγ)² σ₂₁=σ₁₂=(Δβ−Δγ cos θ₁₂)²

Direct calculation shows that the ratio

$${σ}_{a} = \frac{{S}_{12} {S}_{21}}{{S}_{11} {S}_{22}} = \frac{{( Δβ - Δγ cos {θ}_{12} )}^{4}}{{( Δβ - Δγ )}^{4}} \frac{H ( f , {θ}_{12} ; {θ}_{t1} ) H ( f , {θ}_{12} ; {θ}_{t2} )}{H ( f , 0 ; {θ}_{t1} ) H ( f , 0 ; {θ}_{t2} )} \qquad ( 11 )$$

is independent of the beam form and cumulative power absorption along Beam 1 (103) and Beam 2 (104). The frequency variation of the ration σₐ contains information on the scatterer size, and σₐ will also contain information on the degree of anisotropy of the scatterers in the overlap region 103.

For scatterers that are much smaller than the wavelength, we have H=1. For Θ₁₂=π/2 we get

$${σ}_{a} = \frac{{Δβ}^{4}}{{( Δβ - Δγ )}^{4}} = \frac{1}{{( 1 - Δγ / Δβ )}^{4}} \qquad ( 12 )$$

Typical values are |Δβ|˜0.3 and |Δγ|˜0.1, where Δβ and Δγ have opposite signs. This gives gives σₐ˜0.3. In the above situation, we can calculate
Δγ/Δβ=1−σₐ^{-1/4} (13)

As the scatterer dimensions become comparable to or larger than the wave length, the shape of the scatterers influences the scattering cross section so that the H's in Eq.(11) are different from unity. This influences the frequency variation of σₐ, and σₐ becomes dependent on the direction of the incident and the receive beams. For unidirectional fibrous scatterers, like collagene or muscle fibers, one can get large σₐ when the angle of Beam 1 to the fiber direction is similar to the angle of Beam 2 to the fiber direction. The value of σₐ then also increases above 0.3.

When Beam 1 (103) and Beam 2(104) have the same shape and crosses through tissue with similar absorption and scattering, we get A₂˜A₁ and we can calculate S₁₂/S₁₁ or S₁₂/S₂₂ as a measure of the tissue scattering anisotropy.

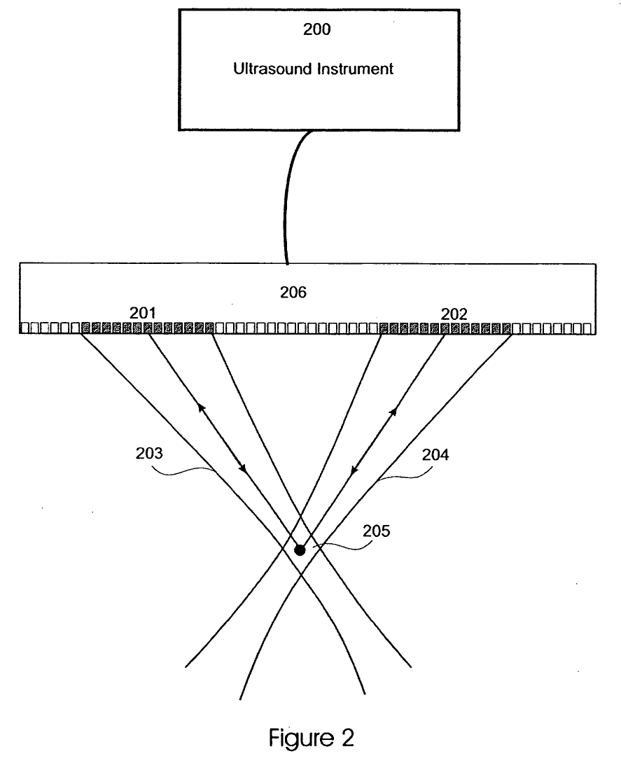

The basic method is conveniently implemented with a linear array as illustrated in FIG. 2, where 206 shows the linear array that is connected to the ultrasound instrument 200. The instrument is designed for free selection of the transmit apertures, where the Figure by way of example illustrates two transmit/receive apertures, Aperture 1 as 201 and Aperture 2 as 202, where one by time delay of the element signals steers the direction of the Beam 1 (203) from Aperture 1 and the direction of Beam 2 (204) from Aperture 2, so that we get an overlap region 205 between the beams. By transmitting and receiving from the two apertures in the same way as for the two transducers in FIG. 1, one can calculate the anisotropy scattering coefficient σₐ as defined in Eq.(3).

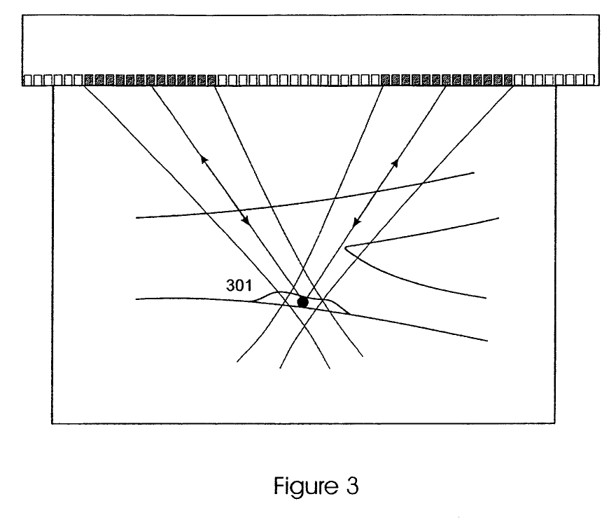

The linear array allows common lateral scanning of Beam 1 and Beam 2 that enables spatial imaging of σₐ, as illustrated in FIG. 3 for noninvasive imaging of a carotid plaque 301. Maintaining the distance between Aperture 1 and Aperture 2 and scanning the beams laterally with a fixed direction angle of the beams, one obtains a spatial image of σₐ at a fixed beam overlap depth. This overlap depth can be varied by varying the distance between Aperture 1 and Aperture 2, with constant direction angles of the beams. The angles between the beams can also be varied for more details of the anisotropic scattering. The two-dimensional back scatter image will indicate the location of the plaque, and one can hence use the back scatter image to limit the spatial region that is actual for interrogation or observation of anisotropic scattering structures by the methods presented above, hence reducing the time of interrogation and the possibility of using multiple directions of the transmit beam with adequate frame rate of the anisotropic scattering imaging.

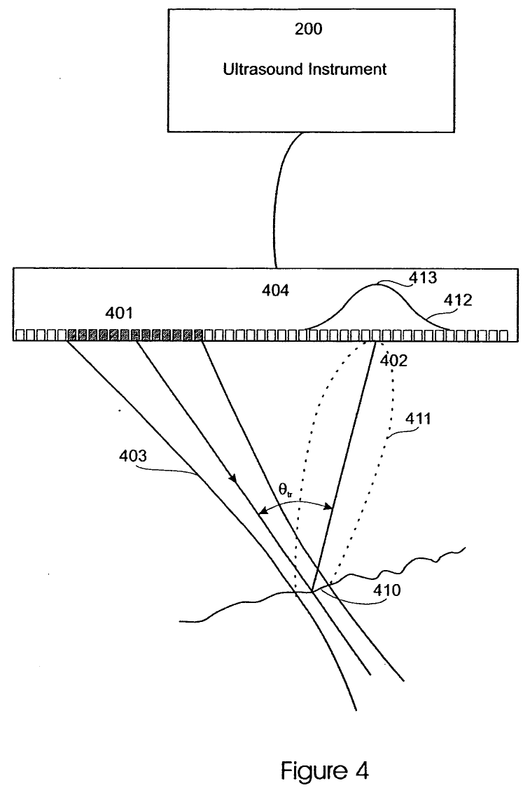

Varying the direction angles of the beams, will in principle also vary all the scattering coefficients. FIG. 4 illustrates a method for at least semi-automatic observation of anisotropic scattering structures of unknown direction in relation to the transducer array. The Figure shows a linear array 404, and an anisotropic scattering structure 410. A pulsed beam 403 is emitted from the aperture 401 of the array, and the scattered wave is picked up by an array element or group of array elements 402, for several positions along the array. This gives an angle θₜᵣ between the transmit and the receive beam directions, that for the given transmit beam varies with the position of the receive elements along the array.

The received signal power as a function of receive element position is monitored at a time interval after the pulse transmission that selects a particular depth along the transmitted beam. An anisotropic scattering structure like 410 will back scatter the main energy in a particular direction indicated by the scattering diagram 411. This scattering diagram produces a variation of the power in the received signal along the element position, indicated as 412 in FIG. 4. Hence, a peaked variation of the scattering power along the element position like at 413 indicates an anisotropic scattering structure at the particular depth, and also the anisotropy direction of the scattering structures. By calculating the power of the received element signals for several time intervals, one can interrogate several depths along the transmitting beam for imaging possible anisotropic scatterers along the transmitted beam. Lateral scanning of the transmit beam then provides a two-dimensional image of anisotropic scattering structures in the tissue. Using several direction angles of the transmitted beam provides possibility of imaging a larger spread of the anisotropy directions.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results, are within the scope of the invention. Moreover, it should be recognized that structures and/or elements and/or method steps shown and/or described in connection with any disclosed form or embodiment of the invention, may be incorporated in any other disclosed or described or suggested form or embodiment as a general matter of design choice. It is the intention, therefore, to be limited only as indicated by the scope of the claims appended hereto.

1. A method for measureing at least one of the magnitude and the frequency dependency of ultrasound scatterer parameters in an observation region of soft tissue, which is insensitive to ultrasound absorption, where
one or more pulsed ultrasound beams are transmitted towards the tissue under investigation and the scattered signal from at least one observation region and at least one reference region is received, where
said observation regions consist of scatterers to be characterized, and said reference regions consist of scattererers with adequately known scattering characteristics so that the signal from said reference regions can be used as reference,
said scatterer parameters in said observation region are obtained as a combination of the received signal from said observation regions and said reference regions.

2. A method for measuring at least one of the magnitude and the frequency dependency of ultrasound scatterer parameters in an observation region of soft tissue according to claim 1, where the scatterers in said reference regions are erythrocytes in blood.

3. A method for measuring at least one of the magnitude and the frequency dependency of ultrasound scatterer parameters in an observation region of soft tissue according to claim 2, where the signal from the scatterers in said reference regions is retrieved from stationary noise by transmitting multiple pulses along essentially the same beam direction and high pass filtering the received signal from said reference regions along the pulse number coordinate, to attenuate the close to stationary noise before the signal from said reference regions is used for said combination with the signal from said observation regions.

4. A method for measuring at least one of the magnitude and the frequency dependency of ultrasound scatterer parameters in an observation region of soft tissue according to claim 1, where the scatterers in said reference regions are parenchymatic cells like in the liver or a gland.

5. A method for measuring at least one of the magnitude and the frequency dependency of ultrasound scatterer parameters in an observation region of soft tissue according to claim 1, where the scatterers in said reference regions are fat cells in atheroma.

6. A method for measuring at least one of the magnitude and the frequency dependency of ultrasound scatterer parameters in an observation region of soft tissue according to claim 1, where the scatterers in said reference regions are contrast agent micro bubbles injected in the blood or other body fluids.

7. A method for measuring at least one of the magnitude and the frequency dependency of ultrasound scatterer parameters in an observation region of soft tissue according to claim 6, where the signal from said contrast agent bubbles is separated from surrounding tissue signal through one of harmonic imaging, and pulse inversion imaging, and manipulation of the scattering properties by a low frequency pulse.

8. A method for measuring at least one of the magnitude, the frequency dependency, and the spatial anisotropy of scatterer parameters in an observation region of soft tissue, where
a 1^{st} and a 2^{nd} ultrasound transducer area is used to generate transmit and receive beams along a 1^{st} and a 2^{nd} beam directions, and where said 1^{st} and a 2^{nd} beam directions intersects to define said observation region, and where
a 1^{st} ultrasound pulse is transmitted from said 1^{st} transducer area along said 1^{st} beam direction, and a 1^{st} receive signal power scattered from said 1^{st} pulse from said observation region along said 1^{st} beam direction is measured at said 1^{st} transducer area, and a 2^{nd} receive signal power scattered from said 1^{st} pulse from said observation region along said 2^{nd} beam direction is measured at said 2^{nd} transducer area, and
a 2^{nd} ultrasound pulse is transmitted from said 2^{nd} transducer area along said 2^{nd} beam direction, and a 3^{rd} receive signal power scattered from said 2^{nd} pulse from said observation region along said 2^{nd} beam direction is measured at said 2^{nd} transducer area, and a 4^{th} receive signal power scattered from said 2^{nd} pulse from said observation region along said 1^{st} beam direction is measured at said 1^{st} transducer area, and where
scatterer parameters in said observation region that is not influenced by the absorption in the tissue, is obtained through a combination of said 1^{st}, 2^{nd}, 3^{rd}, and 4^{th} received signal powers.

9. A method for measuring at least one of the magnitude, the frequency dependency, and the spatial anisotropy of scatterer parameters in an observation region of soft tissue according to claim 8, where said transducer areas are composed of array elements, so that selection of said observation region electronically controlled in an ultrasound instrument through delay direction steering of the said 1^{st} and 2^{nd} beam directions.

10. A method for measuring at least one of the magnitude, the frequency dependency, and the spatial anisotropy of scatterer parameters in an observation region of soft tissue according to claim 8, where said 2 ultrasound transducer areas are 2 separate array transducers.

11. A method for measuring at least one of the magnitude, the frequency dependency, and the spatial anisotropy of scatterer parameters in an observation region of soft tissue according to claim 8, where said 2 ultrasound transducer areas are formed as sub groups of the elements of a linear array and intersection of said beams is electronically controlled in an ultrasound instrument.

12. A method for measuring at least one of the magnitude, the frequency dependency, and the spatial anisotropy of scatterer parameters in an observation region of soft tissue according to claim 8, where said transducer areas are moved along the array surface in order to estimate angular variation of scattering parameters.

13. A method for imaging of spatial variation of at least one of the magnitude, the frequency dependency, and the spatial anisotropy of scatterer parameters in an image region of soft tissue, where
the scattering parameters in selected observation regions are measured according to claim 1 and
the image of the spatial variation of said scattering parameters are obtained by moving said observation region around in said image region.

14. A method for imaging of spatial variation of at least one of the magnitude, the frequency dependency, and the spatial anisotropy of scatterer parameters in an image region of soft tissue, where
the scattering parameters in selected observation regions are measured according to claim 8 and
the image of the spatial variation of said scattering parameters are obtained by moving said observation region around in said image region.

15. A method for characterizing different types of tissue based on measuring at least one of the magnitude, the frequency dependency, and the spatial anisotropy of scatterer parameters obtained according to claim 1.

16. A method for characterizing different types of tissue based on measuring at least one of the magnitude, the frequency dependency, and the spatial anisotropy of scatterer parameters obtained according to claim 8.

17. A method for measuring at least one of the magnitude, the frequency dependency, and the spatial anisotropy of scatterer parameters in an observation region of soft tissue according to claim 11, where said transducer areas are moved along the array surface in order to estimate angular variation of scattering parameters.
